(19) United States
(12) Patent Application Publication
(10) Pub. No.: US 2020/0160957 A1
(43) Pub. Date: May 21, 2020

(54) PRESCRIPTION OUTCOME ENGINE

(71) Applicant: PIONEERRX, LLC, Shreveport, LA (US)

(72) Inventors: Jeffery KEY, Colleyville, TX (US); Joshua HOWLAND, Lewisville, TX (US); Mark BIVINS, Colleyville, TX (US)

(21) Appl. No.: 16/653,945

(22) Filed: Oct. 15, 2019

Related U.S. Application Data
(60) Provisional application No. 62/745,982, filed on Oct. 15, 2018.
(60) Provisional application No. 62/745,621, filed on Oct. 15, 2018.

(51) Int. Cl. G16H 20/10; G16H 10/60; G16H 40/67; G16H 15/00; G06Q 40/08

(52) U.S. Cl. CPC G16H 20/10; G16H 10/60; G06Q 40/08; G16H 15/00; G16H 40/67

(57) ABSTRACT

A prescription outcome system comprising: a prescription outcome engine server connected via a network to a pharmacy computer, the prescription outcome engine server having an engine memory, and an engine processor communicably coupled to the engine memory, and the engine processor configured to execute instructions to: receive claim from a pharmacy, the claim including a patient identifier for a patient and a pharmaceutical identifier for a pharmaceutical desired to be filled; determine if the patent is included on an enrolled patient list, if the pharmaceutical is included on a pharmaceutical allowed list, and if the patient may refill the pharmaceutical at a present time; when the patient is included on the enrolled patient list, the pharmaceutical is included on a pharmaceutical allowed list, and the patient may refill the pharmaceutical at the present time, price the claim, debit employer funds, report adjudication to an employer, report adjudication to a pharmaceutical manufacturer, report adjudication to wholesaler, and return the claim to the pharmacy; and receive an eCare plan from the pharmacy, wherein the eCare plan has patient health data; evaluate the eCare plan to determine if a guaranteed patient health outcome has been achieved; when the engine processor determines the guaranteed patient health outcome has not been achieved, the engine processor directs some or all of the employer funds to be returned to the employer.

### CROSS REFERENCE TO RELATED APPLICATIONS/PRIORITY

The present invention claims priority to United States Provisional Patent Application Nos. 62/745,621 and 62/745,982, both filed Oct. 15, 2018, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### BACKGROUND

A problem the inventors are trying to solve is that manufacturers want to offer a different mechanism for selling their drugs through pharmacies. Today, most drugs in the pharmacy are sold either in cash or insurance companies (Pharmacy Benefit Managers or PBM). Perceptions in the pharmacy that are not cash are typically adjudicated in real time. So when a patent goes into a pharmacy, the patient brings his insurance card, and the pharmacy puts that information in. It goes out through the Internet to a thing called a switch. It comes back with how much it is going to pay and whether or not the patient is eligible and then how much the patient will have to pay. And that's called adjudication.

And that goes through a PBM, a pharmacy benefit management company.

Today that goes through a switch. There are two main switches in the country that are attached to most of the PBMs. Those switches are primarily eRx and Relay Health. Most all PBMs are connected to those two switches. The PBMs might negotiate a discount or rebate from a drug manufacturer in order for the manufacturer to have their product in PBM's formulary. Pharmacies make their money on margin on the difference between what they buy the drugs for and sell the drugs for. Pretty much the PBMs tell the Pharmacies what they are going to sell the drugs for. Branded manufacturers are trying to get the best price for their products in a complex system. A properly designed system and method could dramatically reduce costs for employers, lead to greater availability of drugs for employees and to direct health benefits for the employees. For the foregoing reasons, there is a pressing, but seemingly irresolvable need for such a system and method.

### SUMMARY

Wherefore, it is an object of the present invention to overcome the above mentioned shortcomings and drawbacks associated with the current technology.

The present invention is directed to methods and prescription outcome systems comprising a prescription outcome engine server connected via a network to a pharmacy computer, the prescription outcome engine server having an engine memory, and an engine processor communicably coupled to the engine memory, and the engine processor configured to execute instructions to receive claim from a pharmacy, the claim including a patient identifier for a patient and a pharmaceutical identifier for a pharmaceutical desired to be filled; determine if the patent is included on an enrolled patient list, if the pharmaceutical is included on a pharmaceutical allowed list, and if the patient may refill the pharmaceutical at a present time; when the patient is included on the enrolled patient list, the pharmaceutical is included on a pharmaceutical allowed list, and the patient may refill the pharmaceutical at the present time, price the claim, debit employer funds, report adjudication to an employer, report adjudication to a pharmaceutical manufacturer, report adjudication to wholesaler, and return the claim to the pharmacy; and receive an eCare plan from the pharmacy, wherein the eCare plan has patient health data; evaluate the eCare plan to determine if a guaranteed patient health outcome has been achieved; when the engine processor determines the guaranteed patient health outcome has not been achieved, the engine processor directs some or all of the employer funds to be returned to the employer. According to an additional embodiment the patent health data is acquired via medical testing devices at the pharmacy and the data is automatically incorporated into the eCare plan via a computer network, with the eCare plan being stored on the outcome engine server. According to an additional embodiment the patent health data is acquired at a patient's home via medical testing devices and the data is automatically incorporated into the eCare plan via a computer network, with the eCare plan being stored on the outcome engine server. According to an additional embodiment when the engine processor determines the guaranteed patient health outcome has been achieved, the engine processor creates outcome success reports and directs the reports to be sent, via computer networks, to an employer computer, a pharmacy computer, a wholesaler computer, and a manufacturer computer. According to an additional embodiment, the system further comprises the success report to the wholesaler includes instructions to submit a bonus payment to the pharmacy. According to an additional embodiment, the system further comprises the success report to the manufacturer includes instructions to submit a bonus payment to the pharmacy through the wholesaler. According to an additional embodiment the system further comprises the success report to the pharmacy includes notifications of success and a bonus payment to be paid to the pharmacy. According to an additional embodiment the system further comprises the success report to the employer notice of employee success. According to an additional embodiment, the system further comprising the success report to the wholesaler includes instructions to submit a bonus payment to the pharmacy, the success report to the manufacturer includes instructions to submit a bonus payment to the pharmacy through the wholesaler, the success report to the pharmacy includes notifications of success and a bonus payment to be paid to the pharmacy, and the success report to the employer notice of employee success. According to an additional embodiment, the system further comprising a local switch that receives claims from the pharmacy, the local switch having a switch memory, and a switch processor communicably coupled to the switch memory, and the switch processor configured to execute instructions to determine if the claims are for pharmaceuticals included on the pharmaceutical allowed list, and when the pharmaceutical is not included on the pharmaceutical allowed list, reformat the claim with a primary insurance information and send the claim to an intermediary switch. According to an additional embodiment, when the pharmaceutical is included on the pharmaceutical allowed list, the claim is transferred to the outcome engine server. According to an additional embodiment, the system further comprises a patient ID card that has unique patient identification information for a patient primary insurance and a separate outcome engine patient identification information.

The invention is further related to methods and prescription outcome systems comprising a prescription outcome engine server connected via a network to a pharmacy computer, the prescription outcome engine server having an engine memory, and an engine processor communicably coupled to the engine memory, and the engine processor configured to execute instructions to: receive claim from a pharmacy, the claim including a patient identifier for a patient and a pharmaceutical identifier for a pharmaceutical desired to be filled; determine if the patent is included on an enrolled patient list, if the pharmaceutical is included on a pharmaceutical allowed list, and if the patient may refill the pharmaceutical at a present time; when the patient is included on the enrolled patient list, the pharmaceutical is included on a pharmaceutical allowed list, and the patient may refill the pharmaceutical at the present time, price the claim, debit employer funds, report adjudication to an employer, report adjudication to a pharmaceutical manufacturer, report adjudication to wholesaler, and return the claim to the pharmacy; and receive an eCare plan from the pharmacy, wherein the eCare plan has patient health data; evaluate the eCare plan to determine if a guaranteed patient health outcome has been achieved; when the engine processor determines the guaranteed patient health outcome has not been achieved, direct some or all of the employer funds to be returned to the employer; wherein at least some of the patent health data is acquired via medical testing devices at the pharmacy and the data is automatically incorporated into the eCare plan via a computer network, with the eCare plan being stored on the outcome engine server; wherein at least some of the patent health data is acquired at a patient's home via medical testing devices and the data is automatically incorporated into the eCare plan via a computer network; wherein when the engine processor determines the guaranteed patient health outcome has been achieved, the engine processor creates outcome success reports and directs the reports to be sent, via computer networks, to an employer computer, a pharmacy computer, a wholesaler computer, and a manufacturer computer; wherein the success report to the wholesaler includes instructions to submit a bonus payment to the pharmacy, the success report to the manufacturer includes instructions to submit a bonus payment to the pharmacy through the wholesaler, the success report to the pharmacy includes notifications of success and a bonus payment to be paid to the pharmacy, and the success report to the employer notice of employee success; wherein the prescription outcome system further comprises a local switch that receives claims from the pharmacy, the local switch having a memory, and a processor communicably coupled to the memory, and the processor configured to execute instructions to: determine if the claims are for pharmaceuticals included on the pharmaceutical allowed list, and when the pharmaceutical is not included on the pharmaceutical allowed list, reformat the claim with a primary insurance information and send the claim to an intermediary switch; wherein when the pharmaceutical is included on the pharmaceutical allowed list, the claim is transferred from the local switch to the outcome engine server; and wherein the prescription outcome system further comprises a patient ID card that has unique patient identification information for a patient primary insurance and a separate outcome engine patient identification information.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIG. 1 is a flowchart of an embodiment of the outcome engine adjudication method according to the present invention;

FIG. 2 is a flowchart of an embodiment of the outcome engine reversal method according to the present invention;

FIG. 3 is a flowchart of an embodiment of the outcome engine outcome success/failure method according to the present invention; and

FIG. 4 is a schematic diagram of a computer or server that is used in the processes of FIGS. 1-3.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40% means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

Turning now to the accompanying drawings, a brief description concerning the various components of the present invention will now be briefly discussed. As can be seen in this embodiment, in the outcome model, value streams to the manufacturer as Employer to Wholesaler to Outcome Engine (also termed RxLocal, and value engine, and Pharmacy Outcome Engine), to Pharmacy to Manufacturer.

Employer:

Employer will make an outcome-based payment. Employer will pay for the drug, and if the outcome is not met, Employer will get a refund. Employer will have a direct contracting relationship with the manufacturer. The manufacturer can use their existing reps that are out casing doctors to create relationships with employers, or can use third party agents to start contracting with employers for value based. Employer submits covered lives data to the Prescription Outcome Engine. Keep up with a list. A lot of the Prescription Outcome Engine is like a PBM. A Fixed rate PBM. Employee marketing toward employees the value-based alternative. “Employee, tell your doctor to prescribe MFC-A's insulin instead of another other manufacture's insulin because this puts us in a program with you trying to get better, and if it fails, then we get a refund.”

Wholesaler:

The wholesaler will receive flat fees for value based drugs instead of margin-based payment. If it is a value based, the wholesaler is just getting the cost for distributing the drug. Wholesaler will administer pharmacy payment/refund. Wholesaler will administer fee-based distribution model. Wholesaler will more efficiently move the drug product.

RxLocal value/Outcome Engine, Prescription Outcome Engine:

Works with preferably flat transaction-based fees (preferably not spread margin). Not a PBM where it is trying to get a margin. Preferably each transaction is going to charge for it. Tracks value/outcome-based bonus model—between employer and manufacturer. Tracks fee-based distribution model—between manufacture and Pharmacy. Claim carve out adjudication—the place where it takes the card and says this is a value based drug, we are going to send it a different way and bill it, or drugs that aren't in the value based list, are going to send it to the patient's normal processor, whether it be caremark or whomever. Claims would come from eRx/relay health just like a switch. A BIN PBM would preferably be registered as a switch for this claim carve out. Taking it and reformatting it. The Prescription Outcome Engine administers employer billing/credits. It acts as a clearinghouse for eCare plans/outcomes data, to prove that the customer met the criteria for drug efficacy.

Pharmacy:

The Pharmacy will preferably move to a fixed dispensing fee plus a performance bonus. They will provide enhanced Clinical Services including adherence and lifestyle programs. They will do point of care testing, to prove out the drugs. The Pharmacy will preferably submit clinical data and results. They will preferably handle patient engagement. The pharmacy will preferably do prescriber engagement toward outcomes value alternative. For example, the pharmacy might ask doctor if it can switch a patient's prescription to Manufacture-As' drug (who participates in the value based model), because Pharmacy has money to help manage the employee's/patient's lifestyle better and to do point of care testing, leading to better outcomes for the doctor's patient.

Manufacturer:

Pharmaceutical manufacture conducts employer sales/contracting. Manufacture will preferably provide education and training materials—they are the ones who created it and did the original clinical trials. Manufacture will preferably price drugs based on delivered value/outcome. Manufactures will mitigate channel margin pressure. Manufacturers will preferably prove clinical value in real world settings.

One aim is to move patient to the Prescription Outcome Engine. Manufacture and employer will have agreement that some of the drugs that the employees take come from the manufacturer. For one of such drugs, there will be a separate path for that drug, it will be through the Prescription Outcome Engine.

Manufactures would like to move to a model where they are paid more for value. The inventors believe a brand manufacturer would like to, say Lilly as an example, go to an employer and say, “We want you to buy our drug for “X,” and we are going to guarantee it is successful. And if it is unsuccessful, we are going to refund you a portion of what you pay. Now we realize it might not have been successful because your employee was not taking his meds right, or some other reason. So we're likely not going to refund you all the money, but we are going to get on the same side of the table as you are. Your employee is taking this in order to get better, in order to possibly reduce your hospitalization, and other healthcare spend, and so we are going to guarantee this.”

In the pharmacy world, this is much harder because everything happens in real time.

In order to accomplish this, the inventors have developed a software engine, or a prescription outcome engine to make all these things possible. What follows are a number of embodiments of the prescription outcome engine, though others are contemplated.

The prescription outcome engine preferably takes care of several different workflows. One of the ones is it that somebody out there should know about this agreement. This software engine needs to know. And so an employee walks into a pharmacy gives the pharmacy his card. This is a card with new numbers. So instead of being his normal caremark PBM card, for example, he has a different set of numbers that identify this patient. Let us say is their Rx local card. RxLocal is one name for the inventors' disclosed prescription outcome engine.

The pharmacy puts the information in, it hits the adjudication/the prescription outcome engine. The prescription outcome engine will look at the customer and ask, who is the employer, what manufacturers do that employer have a value based contract with, and is this prescription for a value based drug. If it is a value based drug on contract with the employee's employer, then the pharmacy is going to charge the employer, preferably via ACH, in real time, and the pharmacy is going to charge the employer for that value based drug and then they are going to report it to the proper pieces, and then they are going to tell the pharmacy how much the patient owes.

If the prescription is not for one of these value based drugs, prescription outcome engine will look up and say, okay who is the patient's other insurance, who is their PBM for everything else? It is going to send it off of the other insurance just like the patient appeared with other insurance's card at the pharmacy. It is going to come back to the prescription outcome engine, and the prescription outcome engine is going to send it back to the pharmacy just as if the patient has walked in with Caremark (for example) card.

The prescription outcome engine is going to want to know for this RxLocal member number, what their regular insurance is, if it is not on the value based, what their value based contracts are. It is going to intercept it. It is going to decide what to do with it, whether to debit the employer or not. And preferably, if the pharmacy cannot debit the employer in real time, the patient cannot get their medications. The pharmacy is preferably guaranteed payment before the patient receives his meds.

If it is not one of these value based drugs, it will send the prescription along the same path that it normally would have taken without the RxLocal outcome engine.

Now the prescription outcome engine is also going to report to the various entities that this happened. It will report to the manufacturer that it happened. It is going to report to the employer that it happened. It is going to report to the wholesaler that it happened. In this model the pharmacy for these value based drugs preferably no longer gets paid on the margin, they get paid a dispensing fee.

The prescription outcome engine is going to report to the wholesaler, “Hey this pharmacy just sold thirty pills of Lilly's drugs at a value based deal,” and the wholesaler is going to refund the pharmacy the price the pharmacy paid for those pills and the wholesaler is preferably going to give the pharmacy a dispensing fee instead. Just a straight fee, fee for service. The prescription outcome engine is going to tell the wholesaler what to do.

The Pharmacy is preferably going to be responsible for the measured outcomes. So let's say diabetes, the pharmacy could test the patient every three months for A1C. And they could report that to the prescription outcome engine, which would preferably report that data back to Lilly. The prescription outcome engine is the trusted engine. If the patient meets his success factor, a bonus payment would preferably go to the pharmacy, and the prescription outcome engine would preferably be the piece to tell the wholesaler. The wholesaler would preferably be the ones that pay the bonus to the pharmacy. Using Lilly as an example, Lilly will pay the wholesaler, the wholesaler will pay the pharmacy.

If the patient/employee success factors are not met, in the time frame, the prescription outcome engine would preferably tell the manufacturer that they have to refund the employer, and that money would preferably flow through the prescription outcome engine.

The pharmacy has an incentive for the outcomes to be met, because if they are met, the pharmacy preferably gets a bonus on top of their initial fee.

The wholesaler preferably manages that reimbursement, because the wholesaler is already taking money from a pharmacy and giving money to the pharmacy. They are taking money from the pharmacy when the pharmacy buys drugs from the wholesaler. They are giving money to the pharmacy in form of rebates. This adds an additional line item to what is already flowing back and forth.

The wholesaler preferably manages the money flow between the manufacturer and the pharmacy just like they are today. The bonus payment, the initial dispensing payment, all that would preferably flow through the wholesaler. The money between the manufacturer and the employer is preferably managed by the prescription outcome engine using debits and credits ACH. There will preferably be a new entity there because money's not flowing between the employer and the manufacturer today.

The pharmacies will preferably administer point of care testing to measure success criteria. Additionally, the Pharmacy would preferably be submitting an eCare plan which includes the medications the patient is on, the patient's adherence to those medications, and background about the patient.

The eCare plan is preferably a point of time snapshot, like a care summery.

The value based measure for a manufacturer preferably is required to be something on the manufacture's drug's leaflet. Typically the FDA does not allow manufacturers to guarantee something that was not in their drug's leaflet. So Lilly could not say to the employer, “We are going to guarantee that is your employees will not get their feet amputated from diabetes.” Because their diabetes drug leaflet does not say it prevents feet amputation. It has to be things that are in the leaflet. But, the leaflet for Lilly's diabetes medication might say that it does help to control blood sugar, so the Pharmacies could measure A1C. If this was, on the other hand, a medicine that controlled blood pressure, the test that the pharmacy could do would be blood pressure. So, for this blood pressure medicine, once every certain amount of time the pharmacy could be required to do an in-store blood pressure test and blood pressure results could be submitted as part of the eCare plan. It would preferably be compiled and compared to success rates by the prescription outcome engine. The value based contract gets measured by the prescription outcome engine and is reported appropriately.

There is the adjudication process. There is the refund process. Looking at the outcome engine, preferably it tracks outcomes based on the bonus model, it tracks the fee based distribution model, it does the claim carve out adjudication, it administers the employer billing and credits, and it is a clearinghouse for eCare plans and outcomes.

These actions are preferably automatically conducted by the technology once the process is started. Because the real time nature of the outcome engine, performing this operation without computer technology is not feasible. The technology makes it possible, because pharmacy adjudication is real-time.

There is an algorithm to decide if a given claim is a value based claim, under this contract and be handled in a first way, or this is not a value based claim, and should be handled in a second way.

The steps to perform this method are preferably stored on nonvolatile memory on a prescription outcome engine server, having a CPU, a database, and connections to the pharmacy, the employer, the wholesaler, and the manufacturer via the internet or other computer network. This method helps to address a problem inherent in the technology of real time adjudication technology.

The prescription outcome engine is preferably a cloud server, connected to pharmacy computer by network/internet.

Today the typical thing is one goes to the PBM. The PBMs does some contracting. One embodiment of this disclosed system, would in effect cut the PBMs out of the mix. Which would likely allow more direct engagement from wholesalers to consumer, instead of having a number of extra middlemen in the process.

The extra middlemen is the industry standard, and this process aids in removing that.

### Alternative Embodiments

May not adjudicate employer immediately, may debit the payer immediately, might debit somebody virtually and the pharmacy gets paid sometime later. There are advantages to debiting the employer directly in real time though. For example, sometimes employers go out of business, which can make collecting on past bills impracticable.

Another embodiment is a PBM offering the prescription outcome engine. For example, Caremark could offer a value based subset of drugs, but that would likely only apply Caremark. In such an embodiment, if a manufacturer like Lilly wanted to do value base with a particular employer and they would have to go to that employer's PBM and say they want them to participate, and so it would go through the switch, and when it got to the PBM, the PBM would just decide, this pays differently. But, the PBM likely would not have any of the pieces already today to say report to the wholesaler, you need to do this differently. And reporting back to Lilly, the drug manufacturer, would say “Hey I have a value based drug, 30 value based drug pills were sold, you need to refund the customer.”

If a manufacturer discounts any PBM more than 23 percent, they have to give that discount to all Medicaid customers. This limits flexibility of manufacturers to discount. An advantage of one embodiment of the prescription outcome engine is by the manufacturer dealing directly with the employer, greater discounts may be available. This is allowed, in one embodiment, by turning this into a service where the pharmacy doesn't make a margin. The pharmacy gets paid for a service. In the same way, in this embodiment, wholesaler will get refunded by the manufacturer as well. And the wholesaler is going to receive a transaction fee rather than a margin.

In one embodiment, an employer, who thinks it is a brilliant process, gets on board by contacting the manufacturer. For the manufacturer, they already have many sales people out casing doctors. The manufacturers could redistribute that resource and start hitting employers. According to another embodiment, a third party sales person that sells these agreements may be engaged by the manufacturer. The manufacturer may also offer these agreements to existing brokers who are also brokering an insurance, but you're carving out a subset of pharmaceutical products for this value base, versus the rest of the contract.

For larger employers that is a lot of self-insurance. This may allow that direct manufacturer layer to do a lot of cost reduction right.

Even the smaller employers are either, they're doing their pharmacy without insurance they're paying for their pharmacy products, and some of them, who are smaller who are buying insurance are using high deductible. So they don't do copay, they are not paying for their own employees' pharmacy bills, but their deductible is $7,500 or so. So this could still be carved out in valued based situation, because they still paying the first $7,500 of it.

Other embodiments include additional or alternative methods for how the employer gets deducted or how the manufacturer gets deducted. There may be additional or alternative methods for the wholesaler is notified.

The decision could be programmed into the software of the pharmacy (store wide server or company wide server), rather than, or additionally to occurring in a switch in the cloud/network. In this embodiment, the pharmacy software looks at the prescription and says, “Hey this is a value based drug, don't even send it to the switch. Send it to our own server, they will do the analysis.” In one embodiment, it is as if the switch is removed and placed on the pharmacy's own software.

A benefit of putting the outcome engine in the switch, though, is that the patient can walk into any pharmacy in the country, present their card, and it would adjudicate, and handle it properly.

In other embodiment, the pharmacy preferably has to have contact with the manufacturer to have a dispensing fee and bonus payment instead, but the manufacturer is not entirely limited to a particular pharmacy system. For larger pharmacy companies, such as Walgreens, for example, it has eight thousand stores, the manufacturer might decide that's enough, I am going to tell all of my employers they have to go to a Walgreens, and I going to run the prescription outcome engine in their software. In this embodiment, there is not necessarily a central server, but under an alternate model where the outcome engine runs on pharmacy servers or distributed pharmacy software.

In alternative embodiments, some of the success or efficacy proof could come from patient devices. For example, if a patient was on asthma medication, there are devices that measure a patient's lung capacity. The manufacturer could then be guaranteeing lung capacity for an asthma patient. The patient could have a device he breaths into at home and the device records that. In one embodiment, that data may be automatically uploaded to the pharmacy. In a further embodiment, that data could be automatically uploaded to the prescription outcome engine itself. This would allow, if desired, for the patient to not go through the pharmacy for point of care for measurement for this medication proof data, the prescription outcome engine would preferably get data from the device. There could also be other devices, such as a blood pressure measurement device for use by the patient at home. Part of the manufacturer's contract, or value proposition with the employer could include the manufacture giving the employer a device for employees/patients that are on one of the manufacture's medications whose efficacy criteria could be measured by the device. The device could be part of this guarantee. The employees would preferably measure the efficacy/success criteria themselves, and the device could report the measurements directly and automatically to the prescription outcome engine. The manufacture could say that if the employee/patients have a given successes average over a given amount of time, then the medication is considered successful, or efficacious.

In other embodiments, the drug may or may not be able to be tested at the pharmacy, and the patient goes to an outside lab, and that outside lab could report the data to the outcome engine. This could be through lab for America or a similar company, for example. And that could be the data that registers that the drug has met or failed its success criteria.

And there is preferably a trusted computer, which here is the prescription outcome engine, saying yes this happened or no it did not. Performing this in an automated manner makes the process much more economically feasible.

Another embodiment is a web service or something is not claim based to do with the decision process. It could require that each participant integrates to that web service. For example, to do it out of a large chain pharmacy, such as Walgreens or QS1, or some specific pharmacy company, the service could be offered to the company, but the company would preferably need to implement APIs and then the pharmacy company could benefit as well, outside of the existing adjudication flows. Could include coding it with each different pharmacy system.

Working with a pharmacy helps some embodiments achieve success, because patients need to take their meds, they need the right combination of meds, and pharmacies help ensure that occurs. With automated devices, such as automated glucose meter readers that report data in real time, the pharmacy can still compile, monitor, and report it. These automated devices are especially effective for adolescents and patients some types of mental disorder where they really need to be monitored in real time by somebody else, so they don't get a life threatening problem. There are additionally pills with miniature RFID tags that the patient can swallow for mentally ill patients that once the stomach acid dissolves the outer coating it lets a signal go, and other devices that aid in drug adherence that are contemplated as being part of this system.

These types of patient testing, would preferably still be reported back to a computer-based outcome engine, which decides whether or not the success criteria have been met or not, and triggers value-based bonus to the pharmacy or refund to the employer.

The refund will be preferably ACHed to the employer, in a similar manner as it deducts. Alternatively, a billing statement can be created, and bill the manufacturer, and issue checks to the employer. Preferably, though, the real time ACH side of the pharmacy is used so the risk of payment is not assumed by the pharmacy. Otherwise, a large accounts receivable could be created. So with ACH deductions, it is easy to ACH deposit.

The initial plan preferably comes through to the outcome engine. If the determination is made that drug is not in the formulary, or a value based item. The outcome engine can, for example, adjudicate back to the second re-insurance, or reject the order. The customer will preferably come in with an RxLocal card with an RxLocal BIN, and a PCM, and a member and group number that matches that contract.

Contract will preferably say this is the normal BIN, PCM, Group number and member number for this person for their Carmark. If it is not on formulary, the outcome engine will change the BIN, PCM, member ID and send that out, just as if it had gone out to caremark to begin with. The prescription outcome engine will get it back from Caremark and then send down to the pharmacy. This may incur a double switch to the outcome engine, but preferably not to the customer. One embodiment of this model to the manufacturer has a transaction fee. The contract with the pharmacy could require an extra penny from the pharmacy for running a value based claim.

The system functions best for branded and higher value drugs, such as, for example, $300 and up a month, to $10,000 a month, for example.

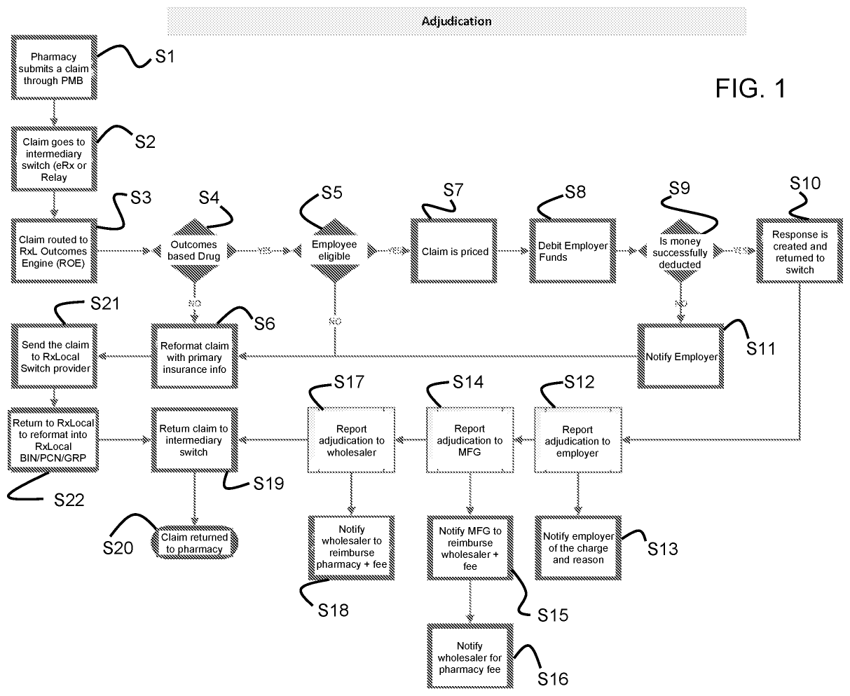

Turning now to FIGS. 1-3, further embodiments of the system and method are described.

In a step S1 the pharmacy submits the claim through a PMB. In step S2 the claim goes to intermediary switch such an eRx or Relay. In step S3 the claim is related to the prescription outcome engine, such as RxL Outcome Engine (ROE). Instep S4 the outcome engine determines if the drug is an outcomes based drug. If the answer is yes, then the process moves to step S5, and the outcome engine determines if the employee is eligible. If the answer in step S4 for was no, the process would move to step as S6 and reformat the claim with primary insurance information. In step S5, if the answer is yes, the process would move to step S7, and the claim is priced. If the answer and step S5 is no, then the process would be to step S6. After the claim is priced at step S7, employer funds are debited in step S8. Next, in step S9, the outcome engine determines if the money was successfully deducted. If yes, in step S10, a response is created and returned to the switch. If the answer to step S9 is no, then the employer is notified in step S11, and the process continues to step S6. After the response is created and returned to the switch in step S10, in step S12, the adjudication is reported to the employer, and the employer is notified of the charge and the reason in step S13. After step S12, the adjudication is reported to the manufacturer by the outcome engine in step S14. The manufacturer is notified to reimburse the wholesaler and transaction fee in step S15, and the outcome engine notifies the wholesaler for the pharmacy fee in step S16. Following step S14, the adjudication is reported to the wholesaler and step S17 with the engine notifying to wholesaler to reimburse the pharmacy and the fee in step S18. After adjudication is reported to the employer, the manufacturer, and the wholesaler and steps S12 through S18, the claim is returned to the intermediary switch in step S19. Then, in step S20 the claim is returned to the pharmacy. If the method passed through step S6, and the claim was reformatted with primary insurance information, and then the claim would be sent two an RxLocal switch provider in step S21. Next, in step S22 the outcome engine would return to RxLocal to reformat into RxLocal BIN/PCN/GRP. Next the outcome engine would return the claim to the intermediary switch in step S19, and then the claim would be returned to the pharmacy in step S20.

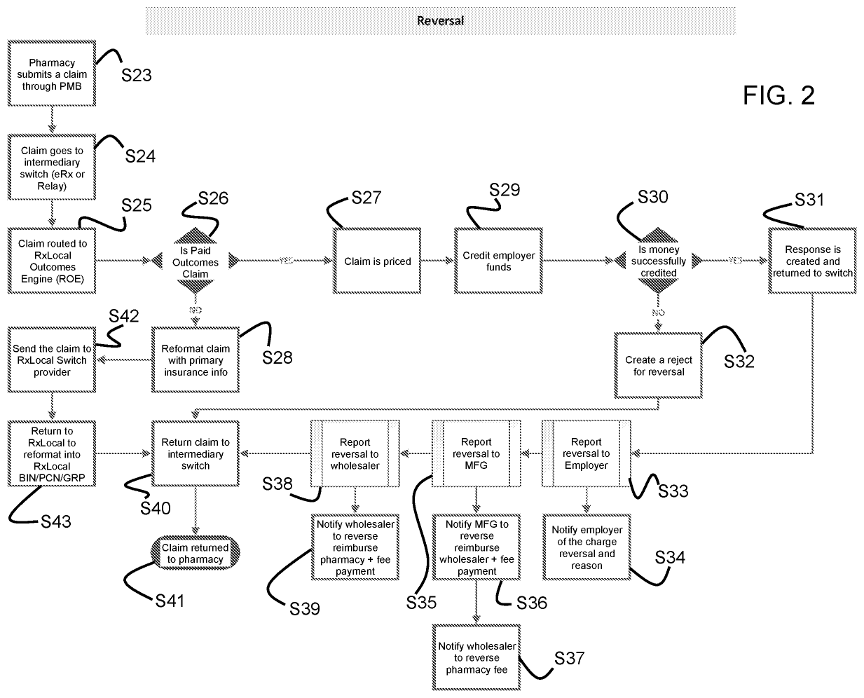

Turning next to FIG. 2, other aspects of the inventive methods, devices and systems are shown. One embodiment of the revearsal process begins when, in step S23, the pharmacy submits a claim through PMB. In step S24, the claim goes to an intermediary switch, such as eRx or Relay. In step S25 the claim is routed to RxLocal outcomes engine (ROE) or another outcome engine as presently claimed. Next, in step S26, the outcome engine determines whether it is a paid outcomes claim? If yes, then the engine precedes to step S27, and the claim is priced. If the outcome engine determines the value in S26 is no, the outcome engine precedes to step S28 and the claim is reformatted with primary insurance information. If the claim is priced in step S27, then, in step S29, employer funds are credited. Then the outcome engine then determine, in step S30, is the money successfully credited? If yes, then in step S31, a response is created and returned to the switch. If no, then the engine precedes from step S30 to S32, and creates a reject for reversal. From S31, the engine precedes to step S33, S35, and S38, reporting the reversal to the employer, the manufacturer, and the wholesaler, respectively. From step S33, the outcome engine notifies the employer of the charge reversal and reason in step S34. From step S35, the outcome engine notifies the manufacturer to reverse reimburse the wholesaler plus the fee payment in step S36, and then notify the wholesaler to reverse the pharmacy fee in step S37. From step S38, the outcome engine notifies the wholesaler to reverse reimburse the pharmacy plus the fee payment in step S39. After step S38, the engine returns to claim to the intermediary switch, in step S40, and then they claim is returned to the pharmacy in step S41.

If the outcome engine determined that the value of step S26 was no, and the process proceeded to step S28, the outcome engine would next send the claim to RxLocal switch provider in step S42. Next, in step S43, the outcome engine would return to RxLocal to reformat into RxLocal BIN/PCN/GRP. Then, the outcome engine would return the claim to the intermediary switch in step S40, and proceed to step S41, where the claim would be returned to the pharmacy.

If the method proceeded through step S32, and the outcome engine created a reject for reversal, then the engine would next return the claim to the intermediary switch in step S40 and then proceeded to return the claim to the pharmacy in step S41.

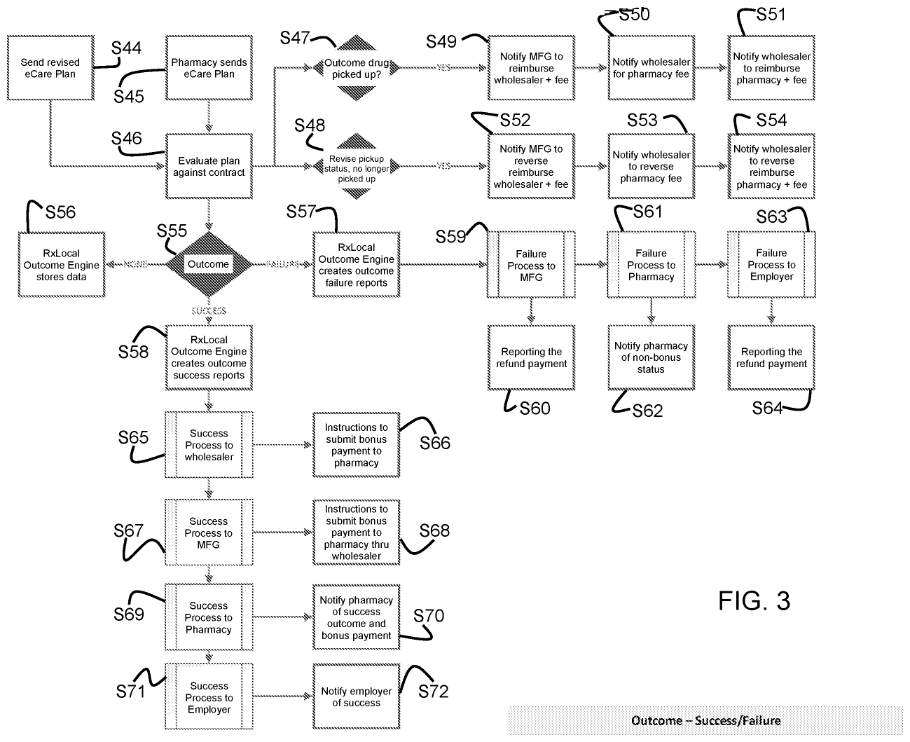

Turning next to FIG. 3, outcome success or failure methods and processes are shown. First, in step S44, a revised eCare plan is sent, and or in step S45 the pharmacy computer sends an eCare plan to the outcome engine server. In step S46, the outcome engine evaluates the plan against a contract, and preferably ask determines three values: first, was the outcome drug picked up, in step S47; second, is there a need to revise pick up status, no longer picked up in step S48; and third, what was the outcome of the measurable, in step S55. In step S47 if the outcome drug was picked up, then the process precedes to step S49, and the outcome engine notifies the manufacturer computer to reimburse the wholesaler plus fee; in step S50 the outcome engine notifies the wholesaler computer for pharmacy fee; and in step S51 notifies the wholesaler computer to reimburse the pharmacy plus the fee. If, in step S48, the outcome engine determines the value of the revise pickup site is coming a longer picked up? is yes, then the outcome engine precedes to step S52, and notifies manufacturer to reverse the wholesaler plus fee, then in step S53, to notify wholesaler reverse the pharmacy fee, and then in step S54, to notify the wholesaler to reverse reimburse the pharmacy and fee.

In the outcome determination step of step S55, if the value is None, that is the outcome was neither a success nor failure, then the outcome engine precedes to step S56, and the RxLocal outcome engine stores the data.

In at the outcome determination step of step S55, if the outcome is failure, as in what guarantees the drug manufacturer made regarding usage of the drug for the employer's employees are not realized, the outcome engine would move to step S57 and the outcome engine creates an outcome failure report. The outcome engine then, in steps S59 S61 and S63, notes a failure process to the manufacturer, the pharmacy, and the employer's computers, respectively. In step S60, the outcome engine reports the refund payment to the manufacturer, in step S62 outcome engine notifies the pharmacy of a non-bonus status, and in step S64, the outcome engine reports a refund payment to the employer.

If the result of the outcome determination in step S55 is success, then in step S58 the outcome engine creates outcome success reports 2 the wholesaler, manufacturer, pharmacy, and employer, and steps s65, S67, S69, and S71. In step S66 the engine sends instruction to the wholesaler computer to submit a bonus payment to the pharmacy. In step 68, the outcome engine sends instructions to the manufacturer to submit bonus payments to the pharmacy through the wholesaler. In step S70, the outcome engine notifies the pharmacy computer of a success outcome and a bonus payment. In step S72, the outcome engine notifies employer engine.

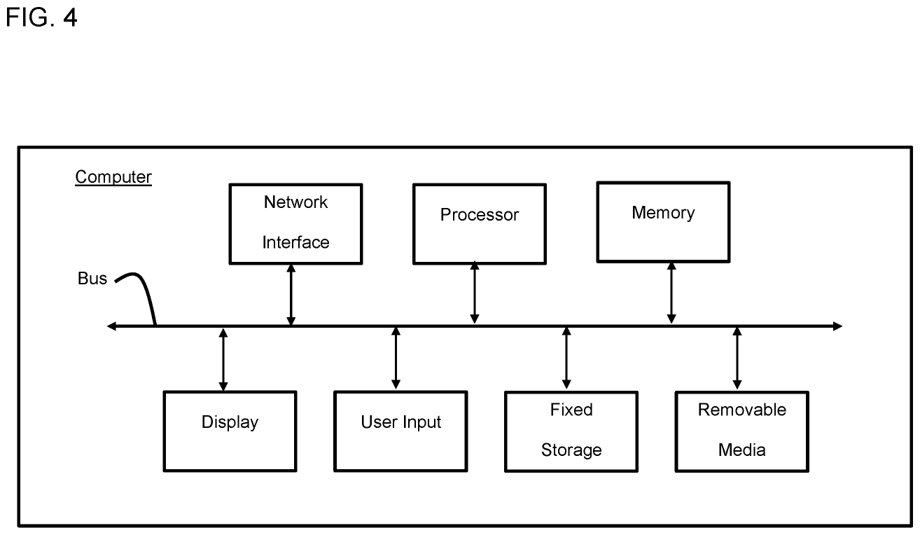

Embodiments of the presently disclosed subject matter may be implemented in and used with a variety of component and network architectures. For example, the system may include one or more computing devices for implementing embodiments of the subject matter described above, including one, some, or all of the outcome engine server, the switch, the pharmacy computer, the manufacturer computer, the warehouse computer, and/or the employer computer. FIG. 4 shows an example of a computing device suitable for implementing embodiments of the presently disclosed subject matter. The computing device may be, for example, a desktop or laptop computer, or a mobile computing device such as a smart phone, tablet, video conferencing/telemedicine system or the like. The computing device may include a bus which interconnects major components of the computer, such as a central processor, a memory such as Random Access Memory (RAM), Read Only Memory (ROM), flash RAM, or the like, a user display such as a display screen, a user input interface, which may include one or more controllers and associated user input devices such as a keyboard, mouse, touch screen (which may be considered part of the interface), and the like, a fixed storage such as a hard drive, flash storage, and the like, a removable media component operative to control and receive an optical disk, flash drive, and the like, and a network interface operable to communicate with one or more remote devices via a suitable network connection.

The bus allows data communication between the central processor and one or more memory components, which may include RAM, ROM, and other memory, as previously noted. Typically, RAM is the main memory into which an operating system and application programs are loaded. A ROM or flash memory component can contain, among other code, the Basic Input-Output System (BIOS) which controls basic hardware operation such as the interaction with peripheral components. Applications resident with the computer are generally stored on and accessed via a computer readable medium, such as a hard disk drive (e.g., fixed storage), an optical drive, floppy disk, or other storage medium.

The fixed storage may be integral with the computer or may be separate and accessed through other interfaces. The network interface may provide a direct connection to a remote server via a wired or wireless connection. The network interface may provide such connection using any suitable technique and protocol as will be readily understood by one of skill in the art, including digital cellular telephone, Wi-Fi, Bluetooth®, near-field, and the like. For example, the network interface may allow the computer to communicate with other computers via one or more local, wide-area, or other communication networks, as described in further detail below.

Many other devices or components (not shown) may be connected in a similar manner (e.g., document scanners, digital cameras and so on). Conversely, all the components shown in FIG. 4 need not be present to practice the present disclosure. The components can be interconnected in different ways from that shown. The operation of a computer such as that shown in FIG. 4 readily known in the art and is not discussed in detail in this application. Code to implement the present disclosure can be stored in computer-readable storage media such as one or more of the memory, fixed storage, removable media, or on a remote storage location.

More generally, various embodiments of the presently disclosed subject matter may include or be embodied in the form of computer-implemented processes and apparatuses for practicing those processes. Embodiments also may be embodied in the form of a computer program product having computer program code containing instructions embodied in non-transitory or tangible media, such as floppy diskettes, CD-ROMs, hard drives, USB (universal serial bus) drives, or any other machine readable storage medium, such that when the computer program code is loaded into and executed by a computer, the computer becomes an apparatus for practicing embodiments of the disclosed subject matter. Embodiments also may be embodied in the form of computer program code, for example, whether stored in a storage medium, loaded into or executed by a computer, or transmitted over some transmission medium, such as over electrical wiring or cabling, through fiber optics, or via electromagnetic radiation, such that when the computer program code is loaded into and executed by a computer, the computer becomes an apparatus for practicing embodiments of the disclosed subject matter. When implemented on a general-purpose microprocessor, the computer program code segments configure the microprocessor to create specific logic circuits.

In some configurations, a set of computer-readable instructions stored on a computer-readable storage medium may be implemented by a general-purpose processor, which may transform the general-purpose processor or a device containing the general-purpose processor into a special-purpose device configured to implement or carry out the instructions. Embodiments may be implemented using hardware that may include a processor, such as a general-purpose microprocessor or an Application Specific Integrated Circuit (ASIC) that embodies all or part of the techniques according to embodiments of the disclosed subject matter in hardware or firmware. The processor may be coupled to memory, such as RAM, ROM, flash memory, a hard disk or any other device capable of storing electronic information. The memory may store instructions adapted to be executed by the processor to perform the techniques according to embodiments of the disclosed subject matter.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I/we claim:

1. A prescription outcome system comprising:
a prescription outcome engine server connected via a network to a pharmacy computer,
the prescription outcome engine server having an engine memory, and an engine processor communicably coupled to the engine memory, and the engine processor configured to execute instructions to:
receive claim from a pharmacy, the claim including a patient identifier for a patient and a pharmaceutical identifier for a pharmaceutical desired to be filled;
determine if the patent is included on an enrolled patient list, if the pharmaceutical is included on a pharmaceutical allowed list, and if the patient may refill the pharmaceutical at a present time;
when the patient is included on the enrolled patient list, the pharmaceutical is included on a pharmaceutical allowed list, and the patient may refill the pharmaceutical at the present time,
price the claim,
debit employer funds,
report adjudication to an employer,
report adjudication to a pharmaceutical manufacturer,
report adjudication to wholesaler, and
return the claim to the pharmacy; and
receive an eCare plan from the pharmacy, wherein the eCare plan has patient health data;
evaluate the eCare plan to determine if a guaranteed patient health outcome has been achieved;
when the engine processor determines the guaranteed patient health outcome has not been achieved, the engine processor directs some or all of the employer funds to be returned to the employer.

2. The prescription outcome system of claim 1 wherein the patent health data is acquired via medical testing devices at the pharmacy and the data is automatically incorporated into the eCare plan via a computer network, with the eCare plan being stored on the outcome engine server.

3. The prescription outcome system of claim 1 wherein the patent health data is acquired at a patient's home via medical testing devices and the data is automatically incorporated into the eCare plan via a computer network, with the eCare plan being stored on the outcome engine server.

4. The prescription outcome system of claim 1 wherein when the engine processor determines the guaranteed patient health outcome has been achieved, the engine processor creates outcome success reports and directs the reports to be sent, via computer networks, to an employer computer, a pharmacy computer, a wholesaler computer, and a manufacturer computer.

5. The prescription outcome system of claim 4, further comprising the success report to the wholesaler includes instructions to submit a bonus payment to the pharmacy.

6. The prescription outcome system of claim 4, further comprising the success report to the manufacturer includes instructions to submit a bonus payment to the pharmacy through the wholesaler.

7. The prescription outcome system of claim 4, further comprising the success report to the pharmacy includes notifications of success and a bonus payment to be paid to the pharmacy.

8. The prescription outcome system of claim 4, further comprising the success report to the employer notice of employee success.

9. The prescription outcome system of claim 4, further comprising the success report to the wholesaler includes instructions to submit a bonus payment to the pharmacy, the success report to the manufacturer includes instructions to submit a bonus payment to the pharmacy through the wholesaler, the success report to the pharmacy includes notifications of success and a bonus payment to be paid to the pharmacy, and the success report to the employer notice of employee success.

10. The prescription outcome system of claim 1 further comprising a local switch that receives claims from the pharmacy, the local switch having a switch memory, and a switch processor communicably coupled to the switch memory, and the switch processor configured to execute instructions to:
determine if the claims are for pharmaceuticals included on the pharmaceutical allowed list, and
when the pharmaceutical is not included on the pharmaceutical allowed list, reformat the claim with a primary insurance information and send the claim to an intermediary switch.

11. The prescription outcome system of claim 10 further comprising when the pharmaceutical is included on the pharmaceutical allowed list, the claim is transferred to the outcome engine server.

12. The prescription outcome system of claim 1 further comprising a patient ID card that has unique patient identification information for a patient primary insurance and a separate outcome engine patient identification information.

13. A prescription outcome system comprising:
a prescription outcome engine server connected via a network to a pharmacy computer,
the prescription outcome engine server having an engine memory, and an engine processor communicably coupled to the engine memory, and the engine processor configured to execute instructions to:
receive claim from a pharmacy, the claim including a patient identifier for a patient and a pharmaceutical identifier for a pharmaceutical desired to be filled;
determine if the patent is included on an enrolled patient list, if the pharmaceutical is included on a pharmaceutical allowed list, and if the patient may refill the pharmaceutical at a present time;
when the patient is included on the enrolled patient list, the pharmaceutical is included on a pharmaceutical allowed list, and the patient may refill the pharmaceutical at the present time,
price the claim,
debit employer funds,
report adjudication to an employer,
report adjudication to a pharmaceutical manufacturer,
report adjudication to wholesaler, and
return the claim to the pharmacy; and
receive an eCare plan from the pharmacy, wherein the eCare plan has patient health data;
evaluate the eCare plan to determine if a guaranteed patient health outcome has been achieved;
when the engine processor determines the guaranteed patient health outcome has not been achieved, direct some or all of the employer funds to be returned to the employer;
wherein at least some of the patent health data is acquired via medical testing devices at the pharmacy and the data is automatically incorporated into the eCare plan via a computer network, with the eCare plan being stored on the outcome engine server;
wherein at least some of the patent health data is acquired at a patient's home via medical testing devices and the data is automatically incorporated into the eCare plan via a computer network;
wherein when the engine processor determines the guaranteed patient health outcome has been achieved, the engine processor creates outcome success reports and directs the reports to be sent, via computer networks, to an employer computer, a pharmacy computer, a wholesaler computer, and a manufacturer computer;
wherein the success report to the wholesaler includes instructions to submit a bonus payment to the pharmacy, the success report to the manufacturer includes instructions to submit a bonus payment to the pharmacy through the wholesaler, the success report to the pharmacy includes notifications of success and a bonus payment to be paid to the pharmacy, and the success report to the employer notice of employee success;
wherein the prescription outcome system further comprises a local switch that receives claims from the pharmacy, the local switch having a memory, and a processor communicably coupled to the memory, and the processor configured to execute instructions to:
determine if the claims are for pharmaceuticals included on the pharmaceutical allowed list, and
when the pharmaceutical is not included on the pharmaceutical allowed list, reformat the claim with a primary insurance information and send the claim to an intermediary switch;
wherein when the pharmaceutical is included on the pharmaceutical allowed list, the claim is transferred from the local switch to the outcome engine server; and
wherein the prescription outcome system further comprises a patient ID card that has unique patient identification information for a patient primary insurance and a separate outcome engine patient identification information.
